(19) United States
(12) Patent Application Publication
(10) Pub. No.: US 2003/0044448 A1
(43) Pub. Date: Mar. 6, 2003

(54) Preservative for grain by-products and processed grain

(76) Inventors: Carol Myers, Norwalk, IA (US); Heath Kellogg, Newton, IA (US)

Correspondence Address: Kent A. Herink, Esq., The Financial Center, Suite 2500, 666 Walnut Street, Des Moines, IA, 50309, US

(21) Appl. No.: 10/052,174

(22) Filed: Jan. 17, 2002

Related U.S. Application Data
(60) Provisional application No. 60/262,883, filed on Jan. 19, 2001.

(51) Int. Cl. A23L 1/27; A61K 35/78

(52) U.S. Cl. 424/439; 424/745; 426/268

(57) ABSTRACT

A preservative for high-moisture organic materials including one or more organic acids combined with either or both a surfactant and an antioxidant. Preferably, the organic acids include propionic acid, acetic acid, benzoic acid, or sorbic acid; the surfactants include propylene glycol, lecithin, lysolecithin, and mono- and diglycerides; the antioxidants include synthetic and natural antioxidants, specifically, TBHQ, citric acid, BHT, BHA, tocopherols, and extracts of rosemary.

This application claims priority to provisional application Serial No. 60/262,883, filed Jan. 19, 2001.

### BACKGROUND OF THE INVENTION

1. Field of the Invention

The invention relates generally to preservatives for grain products having a high water activity or moisture content and, more specifically, a blend of an antioxidant, and/or a surfactant, together with one or more organic acids that is effective at preserving ordinarily highly perishable high-moisture grain products.

2. Background of the Prior Art

Today's agricultural commodities are widely used as starting materials in a variety of manufacturing processes, for example, the manufacture of high-fructose corn syrup, brewing of alcohols, extraction of sugars, citrus juice production, extraction of oils, and the like. A by-product of many such processes is a high-moisture, high-fiber organic material. Such materials have potential commercial value, often as feedstuffs for ruminant animals, but are not fully exploited due to their highly perishable nature. Wet corn gluten feed is a good example. A large amount of it is produced as a by-product of wet corn processing; the domestic market is estimated at 5 million tons per year. It is a suitable component in dairy cattle rations. Unfortunately, it will become unpalatable due to spoilage if not fed within 24 to 48 hrs. This limits the market for the wet corn gluten feed to those dairies located within a radius of the processing facility that permits loading, shipment and feeding of the wet corn gluten feed within the short “shelf life” of the product. The market would be very substantially expanded if the product could be preserved for a week or more. No known method of preservation is known to exist which extends the shelf life of the product by any substantial amount.

Materials having characteristics that make them suitable for preservation by the present invention include organic materials that have a water activity (A_{w}) that supports growth of molds, yeasts and/or aerobic bacteria. While some microbes, such as Clostridium botulinium, stop growing at a water activity A_{w}<0.95, yeasts and molds will continue to grow at a water activity of as little as A_{w} 0.7 to 0.75. Most pathogenic aerobic bacteria stop growing at a water activity of around A_{w} 0.90. Commercial grain products having these characteristics include wet corn gluten feed, distillers dried grains, fuzzy cottonseed, wet and dry brewers grains, cottonseed meal, corn hominy feed, almond hulls, wet and dry sugar beet pulp, canola meal, citrus pulp, rice bran, safflower meal, soybean hulls, food processing waste, and wheat mill run. A great many other products, of course, have such characteristics, including many food products.

Accordingly, there is a need to develop an effective and economical preservative or stabilization agent for high-moisture organic materials, particularly by-products of grain processing.

### SUMMARY OF THE INVENTION

The invention consists of a preservative comprising one or more organic acids combined with either or both an antioxidant and a surfactant. In a preferred embodiment of the invention, the organic acids are selected from the group comprising propionic acid, acetic acid, benzoic acid, citric acid, phosphoric acid, and sorbic acid; the antioxidant is selected from the group comprising natural and synthetic antioxidants, including TBHQ (tertiary butylhydroquinone), BHA (butylated hydroxyanisole), BHT (butylated hydroxytoluene), citric acid, tocopherols, and extracts of rosemary; and the surfactant is selected from the group comprising propylene glycol, lecithin, lysolecithin, and mono- and diglycerides. Either organic acids combined with a surfactant or organic acids combined with an antioxidant are efficacious. The rate of application is dependent on the material being treated. In a preferred embodiment, the blend of organic acids is applied at a rate of between about 5 lbs. and about 30 lbs. per ton; the surfactant is included at a rate of between about 0.001% and about 0.05%; and the antioxidant is included at a rate of between about 0.0005% and about 0.01%.

### BRIEF DESCRIPTION OF THE DRAWINGS

FIG. 1 is a chart of the data summarized and presented in Table 5 on the microbe counts of wet corn distiller's grain without or with treatment by a blend of organic acids, a blend of surfactants, and/or a blend of antioxidants.

FIG. 2 is a chart of the influence of treating wet corn distiller's grain without or with low, medium, or high levels of a preservative composition of the present invention on dry matter intake of finishing steers over time.

### DETAILED DESCRIPTION OF A PREFERRED EMBODIMENT

The present invention involves a preservative comprised of one or more organic acids combined with either or both an antioxidant and a surfactant which, when added to a high-moisture organic material, greatly extends the time over which such materials may be stored prior to spoilage. The present invention is particularly suited for application to by-products of grain processing.

The process of the present invention is described with respect to materials and methods for preserving corn gluten feed with a mixture of propionic acid, acetic acid, benzoic acid, and sorbic acid, together with either a blend of propylene glycol and mono- and diglycerides or an antioxidant blend of citric acid and TBHQ, although it will be clear that modifications of the materials, combinations of materials and rates of inclusion may be made by those skilled in the art to obtain the desired results.

Organic acids are widely used as preservatives in the animal feed industry. Each organic acid has a distinct spectrum of activity against microorganisms, specifically molds, yeasts, and aerobic bacteria, that contribute to spoilage in feeds. Higgins, C. and Brinkhaus, F. Efficacy of several organic acids against molds. 1999. J. Applied Poultry Res. 8:480-487. Accordingly, it is usually desirable to use a blend of organic acids in preservative applications so that the spectrum of efficacy of each of the components will complement each other to provide efficacy over a broad range of microorganisms. These efficacies should be taken into account in optimizing the organic acids selected for use when practicing the present invention.

While a blend of citric acid and TBHQ is used in an embodiment of the present invention, this particular blend of antioxidants is not necessary to the practice of this invention. A particularly preferred antioxidant is an extract of rosemary, including the primary active molecules of rosmanol, rosmarinic acid, carnasol, and carnosic acid.

### Experiment 1

The objective of this experiment was to determine which class of ingredients, organic acids, surfactants or antioxidants is essential in controlling microbial growth and heating in wet corn gluten feed (WCGF).

Materials and Methods:

The following treatments were applied to wet corn gluten:

1. Control, untreated

2. Organic acid blend

3. Surfactant blend

4. Antioxidant blend

5. Organic acids+Surfactants

6. Organic acids+Antioxidants

7. Surfactants+Antioxidants

8. Organic acids+Surfactants+Antioxidants

Treatment preparation:

In this experiment, a blend of organic acid components was selected to attempt to obtain broad efficacy against a variety of molds, yeasts and aerobic bacteria. The same is true of the surfactants and antioxidant components.

TABLE 1
Organic Acid Blend

| | % | 700 grams |
|---|---|---|
| Propionic Acid | 86.65 | 606.55 |
| Acetic Acid | 12.03 | 84.21 |
| Benzoic Acid | 1.20 | 8.40 |
| Sorbic Acid | 0.12 | 0.84 |

The organic acid blend was applied at a rate of 150.9 grams per 40 pounds of WCGF (0.89% by weight). Treatment was applied by sprinkling it on while the WCGF was mixing in the Hobart mixer.

TABLE 2
Surfactant Blend

| | % | 10 grams |
|---|---|---|
| Propylene glycol | 28.87 | 2.89 |
| Mono- and di- | 71.13 | 7.11 |
| glycerides | | |

The blend of surfactants was applied at a rate of 1.1 grams per 40 pounds of WCGF (0.00605% by weight). To assure accurate application, the appropriate amount of surfactant blend was added to soybean oil (1.1 grams of surfactant+48.9 grams of soybean oil). The soybean oil/surfactant blend was applied with a transfer pipette while the WCGF was being stirred in the Hobart mixer.

TABLE 3
Antioxidant Blend

| | % | 10 grams |
|---|---|---|
| Citric acid | 13 | 1.3 |
| TBHQ | 87 | 8.7 |

The blend of antioxidants was applied at a rate of 0.52 grams per 40 pounds of WCGF (0.00286% by weight). To assure accurate application, the appropriate amount of antioxidant blend was dissolved in water (0.52 grams antioxidant in 99.48 grams of water). This required some heating and stirring. The treatment was sprayed on while the WCGF was being stirred in the Hobart mixer.

Treatment Application:

Wet corn gluten feed was obtained fresh from Minnesota Corn Processors in Columbus, Nebr., and kept refrigerated until treatments were applied. Forty (40) pounds of WCGF was weighed into the bowl of a large Hobart mixer. The mixer was started and allowed to stir for 5 minutes while each treatment was being applied. After treatment, 12 pounds was weighed into each of 3 replicate 24.7-quart Styrofoam coolers. A double layer of cheesecloth was laid over the top. The coolers were placed on a shelf in a room that was approximately 68-72° F. for 15 days.

After 15 days, the contents of each cooler were hand-mixed then sampled. Each sample was tested for mold, yeast and aerobic bacteria counts.

Results:

TABLE 4
Initial counts of microorganisms

| Aerobic bacteria | >10⁶ |
|---|---|
| Yeast | >10⁶ |
| Mold | <10 |

TABLE 5
Day 15 microorganism counts

| Treatment | Aerobes | Yeast | Mold |
|---|---|---|---|
| Control | 2.0 × 10⁸ | 1.5 × 10⁴ | 1.15 × 10⁸ |
| | 1.5 × 10⁷ | 3.0 × 10⁶ | >10⁷ |
| | 4.5 × 10⁷ | 5.0 × 10³ | 1.05 × 10⁸ |
| | Ave = 8.6 × 10⁷ | Ave = 1.0 × 10⁶ | |
| Organic acid | 5.5 × 10⁶ | <10 | 5.0 × 10⁵ |
| (O) | 2.5 × 10⁶ | <10 | 5.0 × 10⁵ |
| | 2.9 × 10⁸ | 5 × 10⁵ | 3.5 × 10⁶ |
| | Ave = 9.9 × 10⁷ | Ave = 1.7 × 10⁵ | Ave = 1.5 × 10⁶ |
| Surfactants | 5.5 × 10⁷ | 2.5 × 10⁴ | 2.5 × 10³ |
| (S) | 3.2 × 10⁷ | 1.0 × 10⁵ | 1.5 × 10⁴ |
| | 6.0 × 10⁷ | 1.0 × 10⁵ | 5.5 × 10⁴ |
| | Ave = 4.9 × 10⁷ | Ave = 7.5 × 10⁴ | Ave = 7.25 × 10⁴ |
| Antioxidants | 6.0 × 10⁷ | 1.0 × 10⁶ | 5.0 × 10⁶ |
| (A) | 1.5 × 10⁷ | 5.0 × 10⁵ | 1.0 × 10⁵ |
| | 1.0 × 10⁷ | 5.0 × 10⁵ | 5.0 × 10⁵ |
| | Ave = 2.8 × 10⁷ | Ave = 6.7 × 10⁵ | Ave = 1.87 × 10⁶ |
| O + S | 6.0 × 10⁴ | <10 | <10 |
| | 1.5 × 10⁵ | <10 | 50 |
| | 1.5 × 10⁵ | <10 | 45 |
| | Ave = 1.5 × 10⁵ | Ave = <10 | Ave = 32 |
| O + A | 2.5 × 10⁵ | <10 | 45 |
| | 2.5 × 10⁵ | <10 | 100 |
| | 5.0 × 10⁴ | <10 | 15 |
| | Ave = 1.8 × 10⁵ | Ave = <10 | Ave = 53 |
| S + A | >10⁷ | 1.5 × 10⁵ | <10 |
| | >10⁷ | 3.0 × 10⁵ | 1500 |
| | >10⁷ | 2.5 × 10⁵ | 20 |
| | Ave = >10⁷ | Ave = 2.3 × 10⁵ | Ave = 5.1 × 10² |
| O + S + A | 8.0 × 10⁴ | <10 | <10 |
| | 1.0 × 10⁵ | <10 | <10 |
| | 3.0 × 10⁵ | <10 | 10 |
| | Ave = 1.6 × 10⁵ | Ave = <10 | Ave = 3.3 |

NOTE:
Those samples with counts <10 were considered “0” when average counts were calculated.

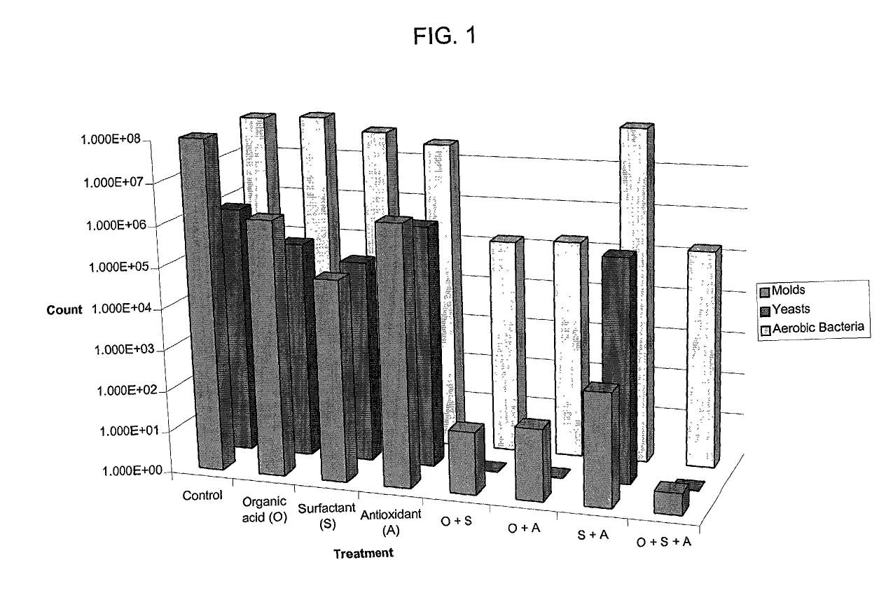

Spoilage of the WCGF was visible in the sample containers. Spoiled WCGF darkened and developed an off odor. Visual and manual observation of the sample containers showed very obvious differences between those samples that were preserved by the experimental treatments, as established by the data summarized and presented in Table 5 (presented graphically in FIG. 1), and those that were not.

The data show that an organic acid is a necessary component of an effective treatment formulation, and that either a surfactant or an antioxidant must be added to the organic acid. Further, a formulation combining all three components, organic acid, surfactant, and antioxidant, was also very effective. The results of the experiment were very surprising. Producers of WCGF had been trying for years to preserve or stabilize the product, without success. The only known treatment involved removing much of the water from the WCGF to reduce its moisture and A_{w} so that it either would no longer support the growth of microorganisms or to the point where conventional preservatives could be used. Removing this quantity of water, however, was time-consuming and relatively expensive. Further, the dried product was less suitable as an animal feed.

### Experiment 2

An experiment was conducted to determine the dose of treatment required to stabilize WCGF. Initial microbial profiles were taken and compared with microbial profiles taken 16 days after treatment. Two levels of antioxidant were used in the treatment formulations.

Materials and Methods

The organic acid blend of Table 1 was buffered by the addition of approximately 15 weight percent inorganic bases and water to improve handling and use with processing equipment, to create what is referred to as Buffered Organic Acid blend (BOA). The surfactant blend of Table 2 and the antioxidant blend of Table 3 were combined in a ratio of 21 g of the surfactant blend to every 10 grams of the antioxidant blend together with 12.35 g of soybean oil to create what is referred to as Surfactant Antioxidant Blend (SAB). Nine formulations were created. Eight of the formulations were designed such that when 5, 10, 15 or 20 lbs/ton of the buffered Organic Acid was applied to WCGF, either 0.05 lbs/ton or 0.1 lbs/ton of the THBQ was also applied. Treatment 9 was the Buffered Organic Acid blend and the Surfactant Antioxidant Blend added at 20 lbs and 0.25 lbs per ton, respectively. See Table 6.

TABLE 6
Treatment Formulations

| | Amount of the BOA | Amount of antioxidant |
|---|---|---|
| Formulation | (lbs/ton) | (lbs/ton) |
| 1 | 20 | 0.05 TBHQ |
| 2 | 15 | 0.05 TBHQ |
| 3 | 10 | 0.05 TBHQ |
| 4 | 5 | 0.05 TBHQ |
| 5 | 20 | 0.1 TBHQ |
| 6 | 15 | 0.1 TBHQ |
| 7 | 10 | 0.1 TBHQ |
| 8 | 5 | 0.1 TBHQ |
| 9 | 20 | 0.25 SAB |
| Control | 0 | 0 |

Wet corn gluten feed was obtained from Minnesota Corn Processors and held at 7° F. for one day until the treatments were applied. Prior to treatment, a sample was tested for initial mold, yeast, and aerobic bacteria counts, as well as moisture and water activity. Treatments 3, 5, 6, 7, and were sprayed on 30 lbs of WCGF while being mixed in a large Hobart mixer for 5 minutes. Ten pounds of WCGF was weighed into each of three 24.7 qt Styrofoam coolers. Treatments 1, 2, 4 and 9 were sprayed on 40 lbs of WCGF while being mixed. Ten pounds was then weighed into each of four coolers. All coolers were covered with a double layer of cheesecloth and held at 72° F. for 16 days. On day 16 three coolers from each treatment were hand mixed and sampled. All samples were tested for mold, yeast and aerobic bacteria counts. The fourth cooler from treatments 1, 2, and 9 was allowed to incubate an additional 17 days, sampled and tested for mold, yeast, aerobic bacteria and water activity. These data are presented in Table 7.

TABLE 7
Dose Titration Study of a Organic Acids, TBHQ and a
Surfactant and Antioxidant Blend on the Stabilization of WCGF

| | Aerobic | | |
|---|---|---|---|
| | Bacteria | Yeast | Mold |
| Treatment | (CFU/g)^{a,b} | (CFU/g)^{a} | (CFU/g)^{a} |
| Initial Counts | 5.0 × 10⁴ | 9.0 × 10³ | 40 |
| 1 | 4.8 × 10⁴ | <10 | <10 |
| 2 | 4.0 × 10⁴ | <10 | <10 |
| 3 | 9.9 × 10⁵ | 4.5 × 10⁵ | <10 |
| 4 | 5.0 × 10⁸ | 3.8 × 10⁵ | 3.3 × 10³ |
| 5 | 4.2 × 10⁴ | <10 | <10 |
| 6 | 3.7 × 10⁴ | 17 | <10 |
| 7 | 1.7 × 10⁷ | 7.0 × 10⁵ | <10 |
| 8 | 6.7 × 10⁸ | 1.3 × 10⁶ | 2.6 × 10⁴ |
| 9 | 8.3 × 10^{9(c)} | 17 | <10 |
| Control | 4.2 × 10⁸ | 6.3 × 10⁵ | 1.7 × 10⁷ |

^{a}Colony forming units per gram
^{b}Average of three replicates
^{c}Replicates varied widely

The initial moisture of the WCGF was 57.73% with a water activity of 0.980. The data in Table 7 indicate that 15 lbs/ton of the Buffered Organic Acid blend with TBHQ included at 0.05 lbs/ton stopped the growth of mold and aerobic bacteria during the 16 day storage period. That treatment level diminished the yeast count from 9.0×10³ CFU/g initially to <10 after 6 days. Higher doses were just as effective. Minimal aerobic bacteria and yeast growth occurred at 10 lbs/ton, while mold growth was completely inhibited. Increasing the inclusion rate of TBHQ at each dose did not improve the effectiveness of the treatment. It must be noted that only the 5+0.05, 5+0.1 and untreated samples were discolored.

### Experiment 3

An experiment was conducted to determine the effects of treating WDGS that has been stored in a silage bag treated at three levels of the composition of the present invention on microbial growth, and dry matter intake and performance of finishing cattle.

Materials and Methods

A treatment formulation was prepared by substituting 1 weight percent TBHQ for 1 weight percent of the propionic acid constituent of the Buffered Organic Acid blend described in Experiment 2. This formulation is referred to as BOA(TBHQ).

One hundred fifty-two (152) steers were weighed and randomly allotted to 8 concrete floor pens with 7 steers/pen and 8 dirt lots with 12 steers/pen at the Southeast Research Farm, Beresford, S. Dak. Steers were adapted to a finishing diet containing 20% wet distiller's grains and solubles (WDGS) on a dry matter basis (Table 8) for 15 days before trial initiation.

TABLE 8
Composition of basal finishing diet

| Item | % in diet DM |
|---|---|
| Wet distiller's grains and solubles | 20.00 |
| Cracked corn | 68.0 |
| Alfalfa hay | 10.0 |
| Mineral supplement | |
| Ground corn | 0.44 |
| Limestone | 0.83 |
| Trace mineralized salt^{a} | 0.70 |
| Rumensin 80 | 0.013 |
| Vitamin A, 30 | 0.002 |
| Copper sulfate | 0.007 |
| Vitamin E | 0.002 |

^{a}Composition (%): Na, >37; Zn, >0.35; Fe, >0.20; Mn, >0.20; Cu, >0.03; I, >0.007; Co, >0.005.

One day before trial initiation, WDGS without or with three levels of BOA(TBHQ) were received and stored in adjacent silage bags. The levels of BOA(TBHQ) were to be 5, 110 and 15 lb/ton of as-is WDGS. Samples of the WDGS for each treatment were collected before being place in silage bags, for later determination of dry matter, crude protein and microbial load (mold and yeast). After adaptation, WDGS without or with BOA(TBHQ) was fed at 20% of the diet dry matter. Treatment diets were fed once daily for 20 days at levels to maintain a 12 bunk score (scattered feed present/most of bottom bunk is exposed). Feed offered was recorded daily. Samples of the major feedstuffs and complete mixed diets were taken weekly, frozen immediately, and later analyzed for percentages of dry matter and crude protein at SDSU, Brookings, S. Dak. A second sample of WDGS for each treatment was collected each week, frozen immediately and later analyzed for microbial load (mold and yeast). Steer weights were taken before feeding on days—15, 0, and 20. On day 21, steers were processed at a commercial processing plant (IBP, Dakota City, Nebr.) and hot carcass weights were recorded. Intake, performance, and carcass data were analyzed using the MIXED Models procedures of SAS (Version 8; Cary, N.C.).

Results

Although it was attempted to treat the WDGS with 5, 10 and 15 lb of BOA(TBHQ) per ton, the actual BOA(TBHQ) levels for the Low, Medium, and High treatments were 4.76, 5.84, and 7.91 lb/ton, respectively. When WDGS was treated with the High level of BOA(TBHQ) (7.9 lb/ton), mold counts were not detected at any of the 3 subsequent sampling dates and yeast count was consistently lower at each of the 3 dates when compared to Control (Table 9).

TABLE 9
BOA(TBHQ) level and it influence on microbial growth on
wet distiller's grains

| | | BOA(TBHQ) level | | |
|---|---|---|---|---|
| Item | Control | Low | Medium | High |
| Actual | 0 | 4.76 | 5.84 | 7.91 |
| BOA(TBHQ) | | | | |
| level, lb/ton | | | | |
| Initial | | | | |
| Mold, CFU/g | <10 | <10 | 1,500 | 150 |
| Yeast, CFU/g | 35,000 | 3,000,000 | 1,150,000 | 800,000 |
| Week 1 | | | | |
| Mold, CFU/g | 800,000 | 400,000 | 370,000 | 0 |
| Yeast, CFU/g | 100,000,000 | 5,000,000 | 5,500,000 | 20 |
| Week 2 | | | | |
| Mold, CFU/g | 100 | 1,400 | <10 | 0 |
| Yeast, CFU/g | 1,050,000 | 30,000 | 0 | 1,350 |
| Week 3 | | | | |
| Mold, CFU/g | 15,000 | 0 | 6,000 | 0 |
| Yeast, CFU/g | 500,000 | 500 | 15,000,000 | 15 |

Treating WDGS with Low and Medium levels of BOA(TBHQ) did not prevent mold and yeast from being detected, but it tended to reduce mold and yeast counts at most of the sampling dates as compared to Control. Mold growth visually appeared to be limited to the surface of the WDGS where exposed to air.

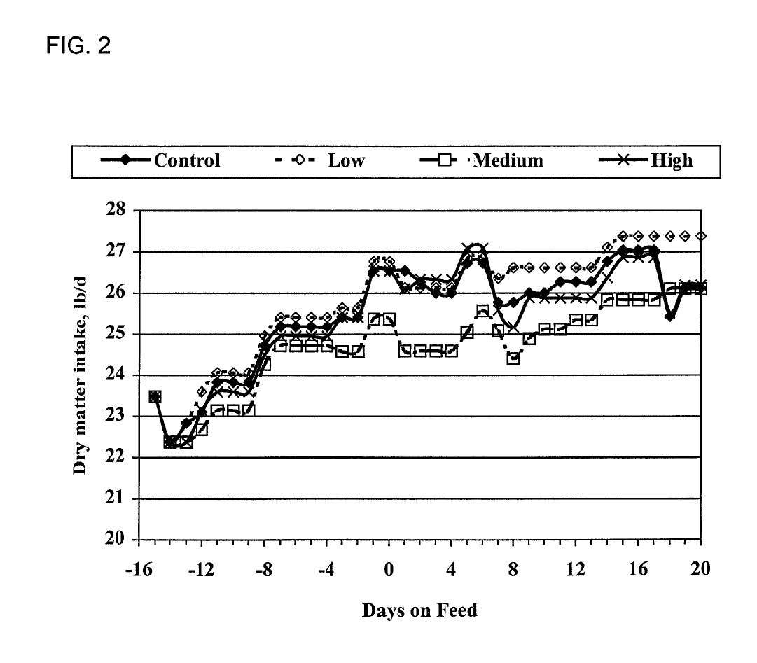

The Control, Low, Medium, and High treatment diets contained 64.2, 63.3, 63.4, and 63.2 percent dry matter, and 15.6, 15.4, 15.2, and 15.0 percent crude protein, respectively. Feeding WDGS treated with Low, Medium, and High levels of BOA(TBHQ) to steers did not have a significant effect on dry matter intake over the 21 day treatment period (FIG. 2). The Medium level of BOA(TBHQ) tended to reduce steer dry matter intake for the first 4 days as compared to the other 3 treatments. This depression in intake may not be the result of BOA(TBHQ) addition, because the intake for 2 of the 4 pens in this treatment group began to decline towards the end of the adaptation period, and continued to be lower for the next 5 days following the diet change. The other 2 pens in the Medium treatment group remained similar to those pens receiving the Control, Low and High treatments. Feeding WDGS without or with BOA(TBHQ) had no effect on steer average daily gain or efficiency of gain (P<0.10); hot carcass weight and dressing percentage also were not influenced by the addition of BOA(TBHQ) (P<0.10) (see Table 10).

TABLE 10
Influence of treating wet distiller's grains without or with
varying levels of BOA(TBHQ) on steer performance,
hot carcass weight, and dressing percent

| | | BOA(TBHQ) level | | | |
|---|---|---|---|---|---|
| Item | Control | Low | Medium | High | SEM |
| Initial weight, | 1162 | 1189 | 1161 | 1163 | 10 |
| lb | | | | | |
| Final weight, lb | 1322 | 1362 | 1325 | 1334 | 12 |
| On test | | | | | |
| Average daily | 4.68 | 4.70 | 4.50 | 4.97 | 0.22 |
| gain, lb/d | | | | | |
| Gain:Feed | 0.177 | 0.175 | 0.176 | 0.188 | 0.007 |
| Hot carcass | 798 | 820 | 801 | 799 | 10 |
| weight, lb | | | | | |
| Carcass dress, | 60.3 | 60.3 | 60.4 | 59.9 | 0.3 |
| % | | | | | |

In conclusion, treating WDGS with levels approaching 8 lb/ton reduced the detection of mold and yeast without having any adverse effects on steer intake and performance.

While not wishing to be bound by any theory, it is believed that the addition of a surfactant to the organic acid component improves the effectiveness of the organic acid at either killing or preventing the growth of microorganisms by improving its dispersal throughout the material being treated, by improving the contact between the organic acid and the microorganisms, by improving the effectiveness of the organic acid at disrupting the transport of water across cell membranes, or combinations of the foregoing. The activity of the antioxidant component is believed to be related to its interference in electron transport necessary for respiration of the microorganisms, particularly in combination with the organic acid and its effect on water transport. Some antioxidants, particularly phenolic antioxidants, are believed to have antimicrobial effects related to their lipohilic properties that allow them to disrupt the cell membrane. A possible explanation of the surprising effectiveness of the present preservative is in the possible synergistic effects of each component's properties at affecting cell membranes and the transport of water and/or nutrients through the membrane.

The foregoing description comprises illustrative embodiments of the present inventions. The foregoing embodiments described herein may vary based on the ability, experience, and preference of those skilled in the art. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

What is claimed is:

1. A composition for preserving a grain product having a water activity greater than 0.70 and initial levels of microbes that cause spoilage of the grain product, comprising:
(a) between about 90 and 99.9 weight percent of one or more organic acids selected from the group comprising propionic, acetic, sorbic, citric, ascorbic, benzoic, and phosphoric acids;
(b) between about 0 and about 10 weight percent of one or more synthetic or natural antioxidants selected from the group comprising TBHQ, citric acid, BHT, BHA, tocopherols, and extracts of rosemary;
(c) between about 0 and about 10 weight percent of one or more synthetic or natural surfactants selected from the group comprising propylene glycol, lecithin, lysolecithin, and mono- and diglycerides; and
(d) wherein the composition is applied to the grain product at between about 0.05 and about 2.5 weight percent to maintain or reduce the level of microbes in the grain product at or below the initial levels for a period of not less than 7 days.

2. A composition as defined in claim 1, wherein the organic acid component comprises one or more acids of the group consisting of propionic, acetic, benzoic, and sorbic acid.

3. A composition as defined in claim 1, wherein animals fed the treated grain product and the untreated grain product consume an equal amount of the treated grain product as the untreated grain product on a dry matter basis.

4. A method for preserving a grain product having a water activity greater than 0.70 and initial levels of microbes that cause spoilage of the grain product, comprising:
(a) selecting between about 90 and 99.9 weight percent of one or more organic acids from the group comprising propionic, acetic, sorbic, citric, ascorbic, benzoic, and phosphoric acids;
(b) selecting between about 0 and about 10 weight percent of one or more synthetic or natural antioxidants from the group comprising TBHQ, citric acid, BHT, BHA, tocopherols, and extracts of rosemary;
(c) selecting between about 0 and about 10 weight percent of one or more synthetic or natural surfactants from the group comprising propylene glycol, lecithin, lysolecithin, and mono- and diglycerides; and
(d) applying the surfactant and/or antioxidant component or components and the organic acid component to a quantity of a grain product wherein the components are applied to the grain product at between about 0.05 and about 2.5 weight percent to maintain or reduce the level of microbes in the grain product at or below the initial levels for a period of not less than 7 days.

5. The method of claim 4, wherein the grain product is selected from the group comprising wet corn gluten feed, wt distiller's grain solubles, distillers dried grains, fuzzy cottonseed, wet and dry brewers grains, cottonseed meal, corn hominy feed, almond hulls, wet and dry sugar beet pulp, canola meal, citrus pulp, rice bran, safflower meal, soybean hulls, food processing waste, and wheat mill run.
